(19) United States
(12) Patent Application Publication
(10) Pub. No.: US 2004/0174954 A1
(43) Pub. Date: Sep. 9, 2004

(54) Medical x-ray device and power module therefor

(76) Inventors: Johan Willem Rust, Eindhoven (NL); Albert Willem Jansen, Eindhoven (NL)

Correspondence Address: Thomas M Lundin, Philips Intellectual Property & Standards, 595 Miner Road, Cleveland, OH, 44143, US

(21) Appl. No.: 10/481,809

(22) PCT Filed: Jun. 28, 2002
(86) PCT No.: PCT/IB02/02483

(30) Foreign Application Priority Data
Jun. 28, 2001 (EP) 01202505.2

(51) Int. Cl. H05G 1/10

(52) U.S. Cl. 378/101

(57) ABSTRACT

The invention concerns a medical X-ray device and a power module therefor. Power module 11 for the X-ray source comprises a substrate formed by one ceramic base plate 12. Several semiconductor devices 13 are attached to the ceramic base plate by means of lead-free solder 16. Cracks in the substrate due to the high thermal load variations are now successfully avoided thus prolonging the lifetime of the power module according to the invention. The invention also refers to a power module according to the invention intended for use in a medical X-ray apparatus.

The present invention relates to a medical X-ray device provided with at least one power module for the X-ray source comprising a substrate, which substrate comprises a ceramic base plate, and one or more semiconductor devices, which are attached to the ceramic base plate by means of solder.

Such a medical X-ray device is known in practice and is currently produced by the applicant. In the known device the substrate further consists of a relatively thick metal back plate onto which the ceramic base plate is attached by means of another layer of solder.

In a medical X-ray device the power module is subjected to high thermal loads. This often leads to cracks in the substrate due to the different thermal expansion coefficients of the different materials of the substrate. As a result thereof the lifetime of the power module is shortened. The lifetime is shortest in medical X-ray devices wherein the thermal loads vary rapidly in time. Some examples of such medical X-ray devices are Computed Tomography devices and C-bow devices, which are frequently started, stopped and restarted during one medical examination.

A second problem is formed by environmental considerations. According to future legislation the solder used should be made of lead-free material. In the known device the use of lead-free solder appears to be impossible, since no lead-free solders suitable for this purpose and having a sufficiently high difference in melting point temperature currently seem to be available.

The object of the invention is to provide a medical X-ray device according to the preamble that solves both problems.

Thereto the medical X-ray device according to the invention is characterized in that the substrate is formed by one ceramic base plate and the semiconductor devices are attached to the ceramic base plate by means of lead-free solder.

The substrate formed by one ceramic base plate has essentially uniform thermal characteristics. Furthermore the use of the other layer of solder to attach the ceramic base plate to the metal back plate becomes redundant. Since cracks mainly arose in that solder layer the lifetime of the power module is now notably prolonged.

It is noted that a power module comprising a substrate, formed by one ceramic base plate with one or more metal layers, and one or more semiconductor devices, which are attached to the ceramic base plate by means of solder, is known per se from U.S. Pat. No. 6,122,170. This patent describes a general purpose power module similar to the one forming part of the state of the art as described above, wherein the metal back plate is left out. However, no mention is made of the use of lead-free solder nor of the specific use in a medical X-ray device imposing the technical problems mentioned above.

According to a first preferred embodiment of a medical X-ray device according to the invention the material of the lead-free solder comprises SnAg, SnAgBiB, SnAgCu and/or all manufacturable alloys thereof. The relevant mechanical and physical properties of these solders make them suitable for the intended use. Thermo-mechanical fatigue life is comparable to that of lead-containing solders. The selected lead-free solders are compatible with most known surface finishes.

According to another preferred embodiment of the medical X-ray device according to the invention the ceramic base plate has a minimal thickness of 3 millimeter. This thickness allows the use of the architecture of the known power module. Depending on the chosen thickness some amount of filler material has to be used.

In yet another preferred embodiment of the medical X-ray device the material of the ceramic base plate comprises AlN, which has allows for good thermal conductivity. As an alternative AlSiO₂ can be used.

The invention also concerns a power module according to one or more of the preceding claims intended for use in medical X-ray apparatus.

The invention will be further explained by means of the attached drawing, in which:

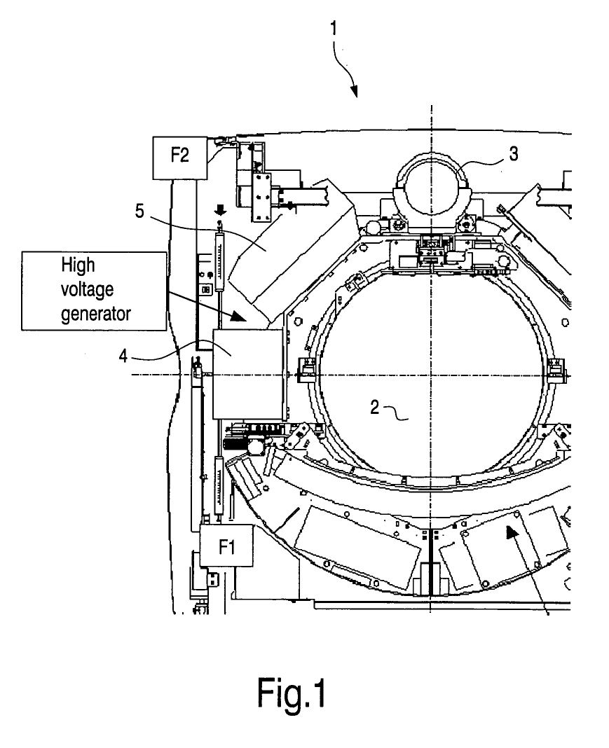

FIG. 1 shows the X-ray device according to the invention schematically in cross section;

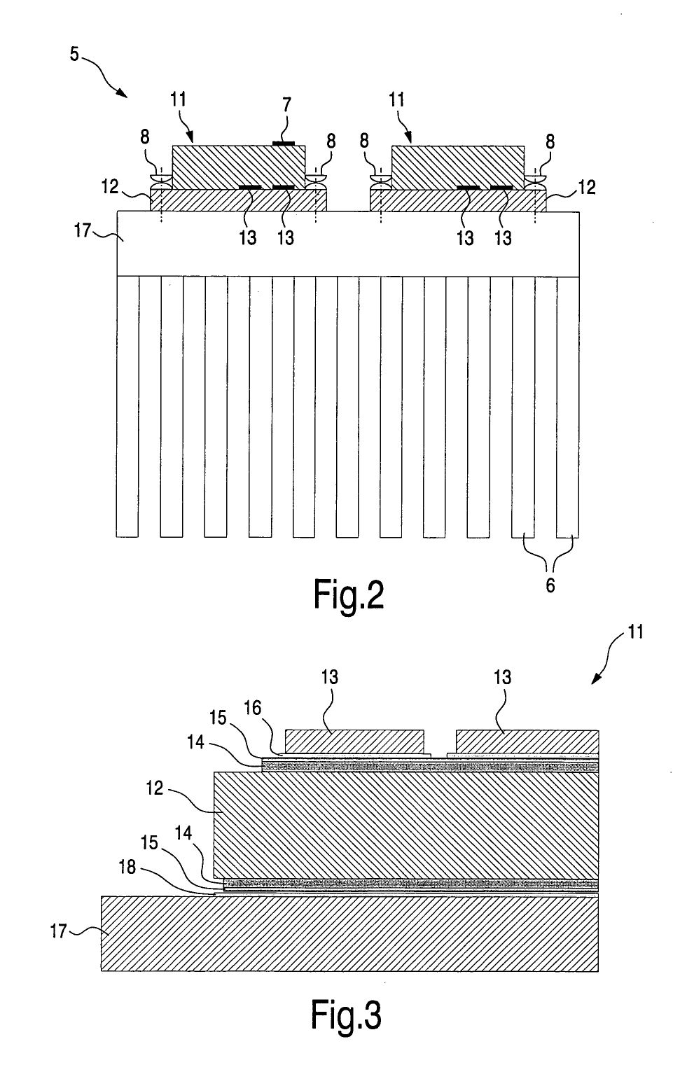

FIG. 2 shows a preferred embodiment of the power module of the medical X-ray device according to FIG. 1 schematically in side view; and

FIG. 3 shows a cross sectional view of a detailed part of the preferred embodiment of the power module according to FIG. 2.

In all figures equal reference numerals are used to denote equal parts.

FIG. 1 schematically shows a cross section of an X-ray device 1 according to the invention. X-ray device 1 is a medical X-ray device provided with a gantry 2 for a subject, usually a human patient, to be examined. An example of such an X-ray device is a Computed Tomography device. The X-ray source of the device 1 comprises an X-ray tube 3, a high voltage tank 4 and an inverter 5.

FIG. 2 schematically shows a side view of part of a preferred embodiment of the inverter 5 incorporating one or more power modules 11 for driving the X-ray source of the medical X-ray device 1. One of the power modules 11 is shown in more detail in cross section in FIG. 3.

FIG. 3 shows part of a power module 11 comprising a substrate formed by a ceramic base plate 12 with metal layers 14 and 15 onto which several semiconductor devices 13 are attached. In the shown example semiconductor devices 13 are preferably IGBT-chips (IGBT=Insulated Gate Bipolar Transistor) for performing DC-AC conversion. The substrate is fixed on a heat radiating plate 17 having appropriate cooling means, such as fins 6 (see FIG. 2). Several means for establishing an electrical connection are provided, such as electrode 7 (see FIG. 2).

Substrate 12 onto which IGBT-chips 13 are attached is electrically isolating thus permitting a large current to be delivered by the front and rear sides of the IGBT-chips 13. For connection to an electrode for this purpose the substrate 12 is provided with a layer 14 of electrically conducting material. Generally layer 14 is a thin film of metal such as copper. For reasons of manufacture layer 14 is present on both sides of the substrate 12.

During operation power module 11 controls large currents. As a result thereof the IGBT-chips 13 dissipate a lot of heat. High demands are therefor put on the radiation characteristics of the power module. For this reason the material of substrate 12 should facilitate heat dissipation and have high thermal conductivity characteristics. Preferably the substrate 12 is made of a ceramic material, preferably AlN. Alternatively AlSiO₂ may be used.

Preferably the thickness of substrate 12 is essentially equal to the thickness of the substrate of the known power module, comprising a metal back plate and a ceramic base plate with one or more intervening layers, such as another solder layer and the electrically conducting layer 14. Common dimensions for the thickness of the known substrate are approximately 5 mm. The thickness for the ceramic base plate 12 should preferably be between 3 and 5 mm. The use of these dimensions allows for the architecture of the remainder of the power module to remain unchanged. If necessary suitable filler material, such as copper strips, can be used. Another advantage of this thickness is that the base plate will be more robust which allows for an asymmetrical set-up with different devices being placed on the base plate, such as chips, diodes etc., all with different dimensions.

The semiconductor devices 13 are attached to the ceramic base plate 12 by means of lead-free solder 16. The material of the lead-free solder comprises preferably SnAg, SnAgBiB, SnAgCu and/or all manufacturable alloys thereof. From a study lead-free wave soldering technology using these solder materials has been found technically viable. Thereto relevant mechanical and physical properties of these solders have been investigated, such as thermal expansion coefficient and melting temperature, which were compared to those of lead-containing solders. Important process parameters, such as solder bath temperature, contact time between board and solder and preheating temperature appear within operable range. More information about these and other suitable lead-free solders can be found in the article “Lead-Free Wave Soldering with VOC-Free Fluxes Part I: Alloy Development Based on SnAgCu, SnBiAg and SnCu, and Process Aspects”, by Biglari et al, published Proceedings IPC-Works 2000, Miami-Florida, Sep. 12, 2000, which article is herein incorporated by reference.

A layer 15 of soldering facilitating material is present between the electrically conducting layer 14 and the lead-free solder 16. Preferably layer 15 comprises Ni which is known in the relevant art for its good wetting characteristics.

Substrate 12 is fixed to heat radiating plate 17 by fixing means 8 having a resilient character. Various suitable fixing means are known in the art, such as clamps or springs. Heat radiating plate 17 comprises a material with high thermal conductivity, e.g. a metal, such as copper or aluminum. Preferably a thin layer 18 of a silicon oil compound or the like is formed to reduce thermal resistance at the interface.

The invention is of course not limited to the described or shown embodiment, but generally extends to any embodiment, which falls within the scope of the appended claims as seen in light of the foregoing description and drawings.

1. Medical X-ray device (1) provided with at least one power module (11) for the X-ray source (3,4,5) comprising a substrate, which substrate comprises a ceramic base plate (12), and one or more semiconductor devices (13), which are attached to the ceramic base plate by means of solder, characterized in that the substrate is formed by one ceramic base plate and the semiconductor devices are attached to the ceramic base plate by means of lead-free solder (16).

2. Medical X-ray device according to claim 1, wherein the material of the lead-free solder comprises SnAg or alloys thereof.

3. Medical X-ray device according to claim 1 or 2, wherein the material of the lead-free solder comprises SnAgBiB or alloys thereof.

4. Medical X-ray device according to claim 1, 2 or 3, wherein the material of the lead-free solder comprises SnAgCu or alloys thereof.

5. Medical X-ray device according to one or more of the preceding claims, wherein the ceramic base plate has a minimal thickness of 3 millimeter.

6. Medical X-ray device according to one or more of the preceding claims, wherein the material of the ceramic base plate comprises AlN.

7. Medical X-ray device according to one or more of the preceding claims, wherein the material of the ceramic base plate comprises AlSiO₂.

8. Power module according to one or more of the preceding claims intended for use in a medical X-ray apparatus.
